(19) United States
(12) Patent Application Publication
(10) Pub. No.: US 2017/0209125 A1
(43) Pub. Date: Jul. 27, 2017

(54) DIAGNOSTIC SYSTEM AND METHOD FOR OBTAINING MEASUREMENTS FROM A MEDICAL IMAGE

(71) Applicant: General Electric Company, Schenectady, NY (US)

(72) Inventor: Sushma Rai, Bangalore (IN)

(21) Appl. No.: 15/004,633

(22) Filed: Jan. 22, 2016

(51) Int. Cl. A61B 8/00; G06F 3/0481; G01B 11/14; G06T 7/00

(52) U.S. Cl. CPC A61B 8/469; G06T 7/0012; G06F 3/04817; A61B 8/466; A61B 8/4427; A61B 8/465; G01B 11/14; G06T 2207/30048; G06T 2207/30204; G06T 2207/20104; G06F 2203/04806

(57) ABSTRACT

Medical diagnostic system includes a user interface having a user screen that is configured to display a medical image to an operator of the diagnostic system. The user interface is configured to show first and second markers on the medical image. The first and second markers represent endpoints of a distance to be measured. The user interface is configured to receive user inputs to move the first marker or the second marker in the ROI. The diagnostic system also includes a controller that is configured to designate a localized section of the medical image that includes the corresponding location of the first marker and display a zoom frame of the localized section over the medical image. The user interface is configured to receive user inputs to move the first marker within the zoom frame to a new location in the ROI within the zoom frame.

### BACKGROUND

The subject matter herein relates generally to medical diagnostic systems and methods for obtaining data relating to a patient's health and/or anatomy, and more particularly, to medical diagnostic systems and methods that are configured to obtain measurements of anatomical matter.

Healthcare providers may use various types of imaging systems to diagnose medical conditions. For example, doctors or other qualified individuals may use an ultrasound imaging system to acquire ultrasound images. It is often desirable to obtain measurements of anatomical matters within the ultrasound images. For example, various measurements of a fetus may be obtained to determine whether the fetus has or will develop certain medical conditions. As another example, doctors may acquire ultrasound images of cardiac structures (e.g., ventricles, atria, valves, septum, and the like) to determine if the patient has a cardiac condition. For instance, cardiovascular mortality and morbidity increases with increasing values of left ventricular (LV) mass. Left-ventricular hypertrophy (LVH) is a thickening of the myocardium of the left ventricle that is also associated with cardiovascular mortality and morbidity.

Conventional user interfaces enable a technician to draw a line from one point to another point on the medical image and determine a distance between the two points. For example, the technician may use a mouse (or similar input device) to drawn the line. For certain ultrasound images or protocols, it is often difficult to identify where the points should be located with respect to the anatomical matter. This challenge has been exacerbated more recently by imaging systems that have become smaller. For example, one ultrasound imaging device includes an ultrasound probe that is connected to a pocket-sized user interface that resembles a flip phone. The user interface includes a user screen. Although this device is effective in obtaining images and measurements therefrom, it can be challenging to draw lines with the device due to the small size of the user screen.

### BRIEF DESCRIPTION

In one embodiment, a medical diagnostic system is provided that includes a user interface having a user screen that is configured to display a medical image to an operator of the diagnostic system. The medical image includes a region-of-interest (ROI) of a patient. The user interface is configured to show first and second markers on the medical image. The first and second markers represent endpoints of a distance to be measured. The user interface is configured to receive user inputs to move the first marker or the second marker to corresponding locations in the ROI. The diagnostic system also includes a controller that is configured to designate a localized section of the medical image that includes the corresponding location of the first marker and display a zoom frame of the localized section over the medical image. The user interface is configured to receive user inputs to move the first marker within the zoom frame to a new location in the ROI within the zoom frame. The controller is configured to measure the distance between the corresponding location of the second marker and the new location of the first marker.

In another embodiment, a method is provided that includes displaying first and second markers on a medical image that has a region-of-interest (ROI) of a patient. The first and second markers represent endpoints of a distance to be measured in the ROI. The first and second markers have corresponding locations in the ROI. The method also includes designating a localized section of the medical image that includes the corresponding location of the first marker and displaying a zoom frame of the localized section over the medical image. The method also includes receiving user inputs to move the first marker within the zoom frame to a new location in the ROI and measuring a distance between the corresponding location of the second marker and the new location of the first marker.

In another embodiment, a medical imaging system is provided that includes a medical imager configured to acquire a medical image of a region-of-interest (ROI) of a patient. The imaging system also includes a user interface having a user screen that is configured to display the medical image to an operator of the medical imaging system. The user interface is configured to display first and second markers on the medical image. The first and second markers represent endpoints of a distance to be measured. The user interface is configured to receive user inputs to move the first marker or the second marker to corresponding locations in the ROI. The imaging system also includes a controller that is configured to designate a localized section of the medical image that includes the corresponding location of the first marker and display a zoom frame of the localized section over the medical image. The user interface is configured to receive user inputs to move the first marker within the zoom frame to a new location in the ROI within the zoom frame. The controller is configured to measure the distance between the corresponding location of the second marker and the new location of the first marker.

### BRIEF DESCRIPTION OF THE DRAWINGS

FIG. 1 illustrates a schematic block diagram of an imaging system in accordance with an embodiment.

FIG. 2 is an illustration of a simplified block diagram of a controller circuit of the imaging system of FIG. 1 in accordance with an embodiment.

FIG. 3 illustrates a hand-carried or pocket-sized ultrasound imaging system.

FIG. 4 illustrates a working window that shows a medical image to an operator of a medical diagnostic system formed in accordance with an embodiment.

FIG. 5 illustrates the working window of FIG. 4 having first and second markers positioned over the medical image.

FIG. 6 illustrates the working window of FIG. 4 in which the first marker is activated by an operator's digit.

FIG. 7 illustrates the working window of FIG. 4 having a zoom frame displayed therein over the medical image.

FIG. 8 illustrates a working window that shows a medical image to an operator of a medical diagnostic system formed in accordance with an embodiment.

FIG. 9 illustrates the working window of FIG. 8 after a zoom frame has been activated by the operator of the diagnostic system.

FIG. 10 illustrates a working window formed in accordance with an embodiment that includes zoom icons.

FIG. 11 illustrates the working window of FIG. 10 after one of the zoom icons has been activated to provide a zoom frame.

FIG. 12 is a flow chart of a method of obtaining a measurement of anatomical matter in accordance with an embodiment.

FIG. 13 illustrates an ultrasound system having a probe that may be configured to acquire ultrasonic data or multi-plane ultrasonic data.

FIG. 14 illustrates an ultrasound imaging system provided on a movable base.

### DETAILED DESCRIPTION

Exemplary embodiments that are described in detail below provide systems and methods for obtaining measurements of a patient from a diagnostic medical image. The patient may be human or animal. In particular embodiments, the medical image is an ultrasound medical image. However, in other embodiments, the medical image may be acquired from one or more other imaging modalities. For instance, various embodiments may be implemented in connection with x-ray imaging systems, magnetic resonance imaging (MRI) systems, computed-tomography (CT) imaging systems, positron emission tomography (PET) imaging systems, or combined imaging systems, among others.

At least some embodiments may be useful for systems that display the medical image on a touch-sensitive screen. It may be difficult for an operator to use his or her digit (e.g., finger, thumb, or stylus) to accurately position a marker at a desired location. As such, embodiments may present a zoom frame that includes a section of the medical image that is magnified. The larger magnification may assist the operator in positioning the marker at the desired location.

At least some embodiments may be useful for systems that display the medical image on a relatively small display area, such as those having a diagonal that is twelve (12) inches (or 30.5 centimeters (cm)) or less. In certain embodiments, the screen size may have a diagonal that is eight (8) inches (or 20.5 cm) or less or six (6) inches (or 15.5 cm) or less. In more particular embodiments, the screen size may have a diagonal that is four and a half (4.5) inches (or 11.5 cm) or less. The relatively small display area may also be described as less than 900 cm², less than 500 cm², less than 250 cm², or less than 100 cm². In smaller embodiments, the user screen may be similar to the user screen of a tablet computer or of a smartphone. At least some embodiments may be particularly useful for systems that include a touch-sensitive screen that has a relatively small display area.

A technical effect for one or more embodiments may include obtaining a measurement of anatomical matter of a patient using less time and/or user actions than known systems. Alternatively or in addition to this, a technical effect for one or more embodiments may include positioning a marker more accurately in systems that include a touch-sensitive screen and/or have a relatively small display area. For example, embodiments may present to the operator of the system a magnified localized section of the medical image within a common display area of the user screen that includes the medical image. The medical image may include a region-of-interest (ROI). The magnified localized section may allow the operator to more accurately select a desired position for the marker with respect to the medical image or the ROI. In some embodiments, the operator of the system may position the marker at the desired location using fewer user actions (e.g., presses, wipes) than known systems and/or without magnifying the entire medical image. In such cases, the user may be less likely to become disoriented or lose his or her bearings with respect to the ROI of the patient.

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

As used herein, “anatomical matter” may be an entire organ or anatomical system or may be an identifiable region or structure within the organ or anatomical system. The measurement may be of a physical structure or include space between physical structures. In particular embodiments, the anatomical matter that is analyzed may include abdominal, urological, cardiac, obstetric, and/or pediatric structures. Non-limiting examples of anatomical matter that may be measured includes an abdominal aorta, urinary bladder, gall bladder, kidney/ureter, liver, spleen, lungs, fetal heart, amniotic fluid, placenta, uterus, aorta, left ventricle, myocardium septum wall, inferior vena cava, mitral valve, and aortic valve.

As used herein, a “diagnostic system” may include a system that is configured to analyze a medical image by enabling the viewer to determine at least one measurement from the medical image. Accordingly, the diagnostic system may be or include a medical imaging system. However, the diagnostic system is not required to be capable of obtaining a medical image. For example, in other embodiments, the diagnostic system is only capable of reviewing a medical image to determine a measurement. The medical image may be received from an imaging system or from a storage system. The diagnostic system may be or include a computer that allows the operator to position markers on the medical image for determining measurements of the anatomical matter. The computer may be a large computing system, a desktop computer, or a portable computer, such as a laptop computer, notebook computer, tablet computer, or smartphone. In particular embodiments, the diagnostic system includes a pocket-sized device that is capable of coupling to an ultrasound probe.

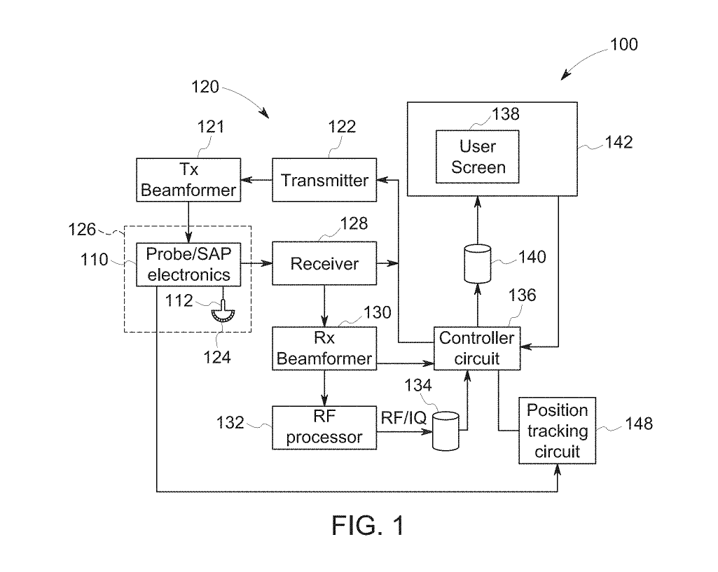

FIG. 1 is a schematic diagram of a medical imaging system 100 in accordance with an embodiment. In an exemplary embodiment, the medical imaging system is an ultrasound imaging system. However, as described herein, embodiments may include other types of imaging systems or may not include any imaging system. The medical imaging system 100 includes an ultrasound probe 126 having a transmitter 122 and probe/SAP electronics 110. The ultrasound probe 126 may be configured to acquire ultrasound data or information from a ROI of the patient. The ROI may include anatomical matter, such as those described above. The ultrasound probe 126 is communicatively coupled to a controller circuit 136 via the transmitter 122. The controller circuit may also be referred to as a “controller” or a “system controller.” The controller circuit may include one or more processors.

The transmitter 122 transmits a signal to a transmit beamformer 121 based on acquisition settings received by the operator. The acquisition settings may define an amplitude, pulse width, frequency, and/or the like of the ultrasonic pulses emitted by the transducer elements 124. The acquisition settings may be adjusted by the user by selecting a gain setting, power, time gain compensation (TGC), resolution, and/or the like from a user interface 142. The signal transmitted by the transmitter 122 in turn drives the transducer elements 124 within the transducer array 112. The transducer elements 124 emit pulsed ultrasonic signals into a patient (e.g., a body). A variety of a geometries and configurations may be used for the array 112. Further, the array 112 of transducer elements 124 may be provided as part of, for example, different types of ultrasound probes.

The transducer elements 124, for example piezoelectric crystals, emit pulsed ultrasonic signals into a body (e.g., patient) or volume corresponding to the acquisition settings. At least a portion of the pulsed ultrasonic signals back-scatter from the ROI or the anatomical matter to produce echoes. The echoes are delayed in time according to a depth, and are received by the transducer elements 124 within the transducer array 112. The ultrasonic signals may be used for imaging, for generating and/or tracking shear-waves, for measuring differences in compression displacement of the tissue (e.g., strain), and/or for therapy, among other uses. For example, the probe 126 may deliver low energy pulses during imaging and tracking, medium to high energy pulses to generate shear-waves, and high energy pulses during therapy.

The transducer array 112 may have a variety of array geometries and configurations for the transducer elements 124 which may be provided as part of, for example, different types of ultrasound probes 126. The probe/SAP electronics 110 may be used to control the switching of the transducer elements 124. The probe/SAP electronics 110 may also be used to group the transducer elements 124 into one or more sub-apertures.

The transducer elements 124 convert the received echo signals into electrical signals which may be received by a receiver 128. The electrical signals representing the received echoes are passed through a receive beamformer 130, which performs beamforming on the received echoes and outputs a radio frequency (RF) signal. The RF signal is then provided to an RF processor 132 that processes the RF signal. The RF processor 132 may generate different ultrasound image data types, such as B-mode, color Doppler (velocity/power/variance), tissue Doppler (velocity), and Doppler energy, for multiple scan planes or different scanning patterns. The RF processor 132 gathers the information (e.g. I/Q, B-mode, color Doppler, tissue Doppler, and Doppler energy information) related to multiple data slices and stores the data information, which may include time stamp and orientation/rotation information, on the memory 134.

Alternatively, the RF processor 132 may include a complex demodulator (not shown) that demodulates the RF signal to form IQ data pairs representative of the echo signals. The RF or IQ signal data may then be provided directly to a memory 134 for storage (e.g., temporary storage). Optionally, the output of the beamformer 130 may be passed directly to a controller circuit 136.

The controller circuit 136 may be configured to process the acquired ultrasound data (e.g., RF signal data or IQ data pairs) and prepare frames of ultrasound image data for display on the user screen 138. The controller circuit 136 may include processing circuitry that is configured to perform one or more tasks, functions, or steps, such as those described herein. For instance, the controller circuit 136 may be a logic-based device that performs operations based on instructions stored on a tangible and non-transitory computer readable medium, such as memory. It may be noted that “processor” or “processing unit,” as used herein, is not intended to necessarily be limited to a single processor or single logic-based device. For example, the controller circuit 136 may include a single processor (e.g., having one or more cores), multiple discrete processors, one or more application specific integrated circuits (ASICs), and/or one or more field programmable gate arrays (FPGAs). In some embodiments, the controller circuit 136 (or a portion thereof) is an off-the-shelf device that is appropriately programmed or instructed to perform operations, such as the algorithms described herein.

Optionally, the controller circuit 136 may include a central controller circuit (CPU), one or more microprocessors, a graphics controller circuit (GPU), or any other electronic component capable of processing inputted data according to specific logical instructions. Having the controller circuit 136 that includes a GPU may be advantageous for computation-intensive operations, such as volume-rendering.

The controller circuit 136 may also be a hard-wired device (e.g., electronic circuitry) that performs the operations based on hard-wired logic that is configured to perform the algorithms described herein. Accordingly, the controller circuit 136 may include one or more ASICs and/or FPGAs.

The controller circuit 136 is configured to perform one or more processing operations according to a plurality of selectable ultrasound modalities on the acquired ultrasound data, adjust or define the ultrasonic pulses emitted from the transducer elements 124, adjust one or more image display settings of components (e.g., ultrasound images, interface components) displayed on the user screen 138, and other operations as described herein. Acquired ultrasound data may be processed in real-time by the controller circuit 136 during a scanning or therapy session as the echo signals are received. Additionally or alternatively, the ultrasound data may be stored temporarily on the memory 134 during a scanning session and processed in less than real-time in a live or off-line operation.

The ultrasound imaging system 100 may include a memory 140 for storing processed frames of acquired ultrasound data that are not scheduled to be displayed immediately or to store post-processed images (e.g., shear-wave images, strain images), firmware or software corresponding to, for example, a graphical user interface, one or more default image display settings, and/or the like. The memory 140 may be a tangible and non-transitory computer readable medium such as flash memory, RAM, ROM, EEPROM, and/or the like.

One or both of the memory 134 and 140 may store 3D ultrasound image data sets of the ultrasound data, where such 3D ultrasound image data sets are accessed to present 2D and 3D images. For example, a 3D ultrasound image data set may be mapped into the corresponding memory 134 or 140, as well as one or more reference planes. The processing of the ultrasound data, including the ultrasound image data sets, may be based in part on user inputs, for example, user selections received at the user interface 142. The ultrasound imaging system 100 may also include a position tracking circuit 148. The position tracking circuit 148 tracks a position of the probe 126 and communicates the position to the controller circuit 136.

The controller circuit 136 is operably coupled to the user interface 142. The user interface 142 may include, among other things, a user screen 138 and/or an input device (not shown). The user screen 138 may also be referred to as a display or display device. The user screen 138 may include one or more liquid crystal displays (e.g., light emitting diode (LED) backlight), organic light emitting diode (OLED) displays, plasma displays, CRT displays, and/or the like. The user screen 138 may display patient information, ultrasound images and/or videos, components of a display interface, one or more 2D, 3D, or 4D ultrasound image data sets from ultrasound data stored on the memory 134 or 140 or currently being acquired, measurements, diagnosis, treatment information, and/or the like received by the user screen 138 from the controller circuit 136.

The user interface 142 controls operations of the controller circuit 136 and is configured to receive inputs from the user. The user interface 142 may include hardware, firmware, software, or a combination thereof that enables an individual (e.g., an operator) to directly or indirectly control operation of the imaging system 100 and the various components thereof. As shown, the user interface 142 includes the user screen 138. The user screen 138 may include one or more separate screens or displays that are oriented to be viewed by the operator. In some embodiments, the user interface 142 may also include one or more input devices (not shown), such as a physical keyboard, mouse, and/or touchpad. In an exemplary embodiment, the user screen 138 is a touch-sensitive display (e.g., touchscreen) that can detect a presence of a touch from an operator of the imaging system 100 and can also identify a location in the display area of the touch. The touch may be applied by, for example, at least one of an individual's hand, glove, stylus, or the like. As such, the touch-sensitive display may receive inputs from the operator and also communicate information to the operator. For embodiments that include touch-sensitive displays, the user screen and the input device may be the same component.

In one or more embodiments, “communicatively coupled” includes devices or components being electrically coupled to each other through, for example, wires or cables and also includes devices or components being wirelessly connected to each other such that one or more of the devices or components of the imaging system 100 may be located remote from the others. For example, the user interface 142 may be located at one location (e.g., hospital room or research laboratory) and the probe 112 (or portions thereof) may be remotely located.

In one or more embodiments, a diagnostic ultrasound image includes an image obtained through an ultrasound imaging system that is based on one or more ultrasound processing techniques (e.g., color-flow, acoustic radiation force imaging (ARFI), B-mode imaging, spectral Doppler, acoustic streaming, tissue Doppler, C-scan, elastography, M-mode, power Doppler, harmonic tissue strain imaging, among others). The ultrasound images may be two-dimensional (2D), three-dimensional (3D), or four-dimensional (4D). An ultrasound image may include only a single image frame or may include a set of image frames. The set of image frames may be a series of image frames obtained over a duration of time. The duration of time may encompass one or more cardiac cycles. In order to distinguish different images or image frames, an ultrasound image (or an image frame) may also be referred to as a reference image (or reference frame), a real-time image (or a real-time frame), a cardiac-cycle image (or a cardiac-cycle frame).

In one or more embodiments, a “user-selectable element” includes an identifiable element that is configured to be activated by an operator. The user-selectable element may be a physical element of an input device, such as a keyboard or keypad, or the user-selectable element may be a graphical-user-interface (GUI) element (e.g., a virtual element) that is displayed on a screen. User-selectable elements are configured to be activated by an operator during a diagnostic session. Activation of the user-selectable element may be accomplished in various manners. For example, the user-selectable element (physical or virtual) may be pressed by the operator, selected using a cursor and/or a mouse, selected using keys of a keyboard, voice-activated, and the like. By way of example, the user-selectable element may be a key of a keyboard (physical or virtual), a tab, a switch, a lever, a drop-down menu that provides a list of selections, a graphical icon, and the like. In some embodiments, the user-selectable element is labeled or otherwise differentiated (e.g., by drawing or unique shape) with respect to other user-selectable elements. When a user-selectable element is activated by an operator, signals are communicated to the imaging system 100 (e.g., the computing system 102) that indicate the operator has selected and activated the user-selectable element and, as such, desires a predetermined action. The signals may instruct the imaging system 100 to act or respond in a predetermined manner.

In some embodiments, the imaging system 100 may be activated by user motions without specifically engaging a user-selectable element. For example, the operator of the imaging system 100 may command the imaging system 100 to shows a zoom frame by quickly tapping the user screen 138, pressing the user screen 138 for longer periods of time, swiping the user screen 138 with one or more fingers (or stylus unit), or pinching the user screen 138 with multiple fingers (or styluses). Other gestures may be recognized by the user interface 142. In other embodiments, the gestures may be identified by the imaging system 100 without engaging the screen. For example, the imaging system 100 may include a camera (not shown) that monitors the operator. The imaging system 100 may be programmed to respond when the operator performs predetermined motions. As yet another example, the user interface 142 may be voice-activated such that the user interface 142 presents, for example, a zoom frame on the user screen 138 when the operator speaks a voice command.

Optionally, the system 100 may constitute a medical diagnostic system that does not include a medical imager, such as the probe 126. In such embodiments, the system 100 may only include, for example, the controller circuit 136, the memory 140, and the user interface 142.

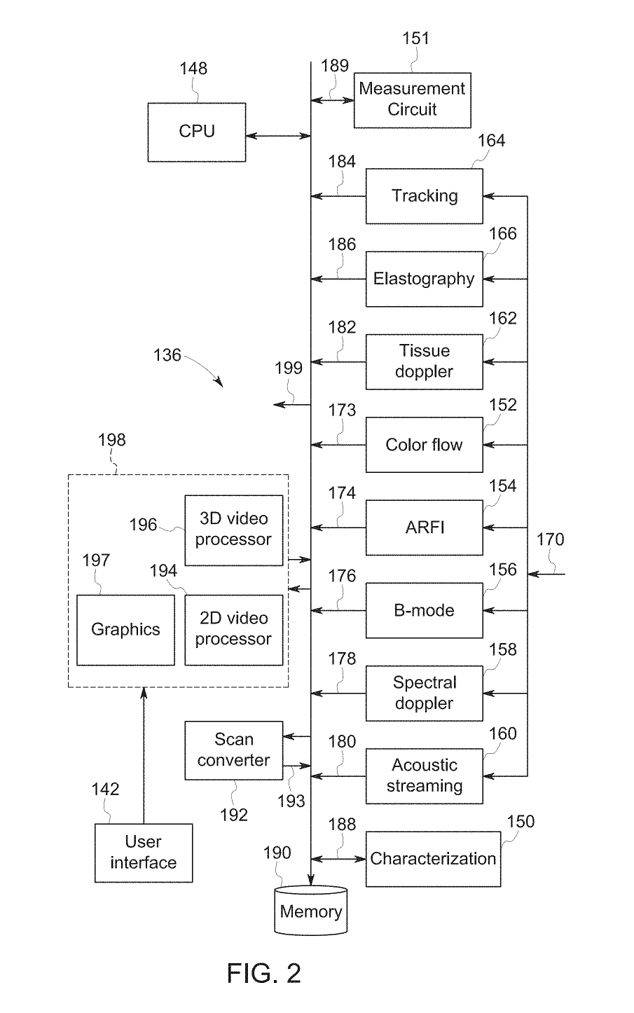

FIG. 2 is an exemplary block diagram of the controller circuit 136. The controller circuit 136 is illustrated in FIG. 2 conceptually as a collection of circuits and/or software modules, but may be implemented utilizing any combination of dedicated hardware boards, DSPs, one or more processors, FPGAs, ASICs, a tangible and non-transitory computer readable medium configured to direct one or more processors, and/or the like.

The circuits 150-166 (e.g., dedicated hardware, micro-processors, software modules) perform mid-processor operations representing one or more visual diagnostics, operations, data manipulation, and/or the like of the ultrasound imaging system 100. The circuits 150-166 may be controlled by the controller circuit 136. The controller circuit 136 may receive ultrasound data 170 in one of several forms. In the embodiment of FIG. 2, the received ultrasound data 170 constitutes IQ data pairs representing the real and imaginary components associated with each data sample. The IQ data pairs are provided to one or more circuits, for example, a color-flow circuit 152, an acoustic radiation force imaging (ARFI) circuit 154, a B-mode circuit 156, a spectral Doppler circuit 158, an acoustic streaming circuit 160, a tissue Doppler circuit 162, a tracking circuit 164, and an elastography circuit 166. Other circuits may be included, such as an M-mode circuit, power Doppler circuit, among others. However, embodiments described herein are not limited to processing IQ data pairs. For example, processing may be done with RF data and/or using other methods. Furthermore, data may be processed through multiple circuits.

Each of the circuits 152-166 is configured to process the IQ data pairs in a predetermined manner to generate, respectively, color-flow data 173, ARFI data 174, B-mode data 176, spectral Doppler data 178, acoustic streaming data 180, tissue Doppler data 182, tracking data 184 (e.g., ROI data acquisition location), elastography data 186 (e.g., strain data, shear-wave data), among others, all of which may be stored in a memory 190 (or memory 134 or memory 140 shown in FIG. 1) temporarily before subsequent processing. The data 173-186 may be stored, for example, as sets of vector data values, where each set defines an individual ultrasound image frame. The vector data values are generally organized based on the polar coordinate system. The memory 190 may be a tangible and non-transitory computer readable medium such as flash memory, RAM, ROM, EEPROM, and/or the like.

A scan converter circuit 192 accesses and obtains from the memory 190, the vector data values associated with an image frame and converts the set of vector data values to Cartesian coordinates to generate an ultrasound image frame 193 formatted for display. The ultrasound image frames 193 generated by the scan converter circuit 192 may be provided back to the memory 190 for subsequent processing or may be provided to the memory 134 or the memory 140. Once the scan converter circuit 192 generates the ultrasound image frames 193 associated with the data, the ultrasound image frames 193 may be stored on the memory 190 or communicated over a bus 199 to a database (not shown), the memory 134, the memory 140, and/or to other processors (not shown).

In connection with FIG. 2, the display circuit 198 accesses and obtains one or more of the ultrasound image frames and display settings stored from the memory 190 or from the memory 134 and/or the memory 140 over the bus 199 to display the ultrasound image concurrently with one or more interface components (e.g., graphical elements, such as marker or zoom icons) onto the user screen 138. The display circuit 198 may receive user input from the user interface 142 selecting one or more ultrasound image frames to be displayed that are stored on memory (e.g., the memory 190) and/or selecting a display layout or configuration.

The display circuit 198 of FIG. 2 may include a 2D video processor circuit 194. The 2D video processor circuit 194 may be used to combine one or more of the frames generated from the different types of ultrasound data. Successive frames of images may be stored as a cine loop (4D images) on the memory 190 or memory 140. The cine loop represents a first in, first out circular image buffer to capture image data that is displayed in real-time to the user. The user may freeze the cine loop by entering a freeze command at the user interface 142. The image frame that appears when the cine loop is frozen may be stored, for example, as a medical image that is to be analyzed by a measurement circuit 151.

Optionally, the display circuit 198 may include a 3D processor circuit 196. The 3D processor circuit 196 may access the memory 190 to obtain spatially consecutive groups of ultrasound image frames and to generate three-dimensional image representations thereof, such as through volume rendering or surface rendering algorithms as are known. The three-dimensional images may be generated utilizing various imaging techniques, such as ray-casting, maximum intensity pixel projection and the like.

The display circuit 198 may include a graphics circuit 197. The graphics circuit 197 may access the memory 190 to obtain groups of ultrasound image frames and the ROI data acquisition locations that have been stored or that are currently being acquired. The graphics circuit 197 may generate working windows that include the medical images of the ROI and one or more graphical representations that are simultaneously displayed by the user screen 138. In some cases, the graphical representations may be positioned over (e.g., overlaid) the medical image of the ROI. The graphical representation may include, among other things, markers, zoom icons, user-selectable elements that are positioned within the working window along with the medical image. The graphical representations may be generated using a saved graphical image or drawing (e.g., computer graphic generated drawing).

The measurement circuit 151 is configured to receive locations of first and second markers relative to a medical image and determine a distance between the first and second markers. The measurement circuit 151 may store a formula or algorithm for determining a distance between the first and second markers when the locations of the first and second markers are known. For example, the measurement circuit 151 may determine the distance based on a size and/or magnification of the medical image.

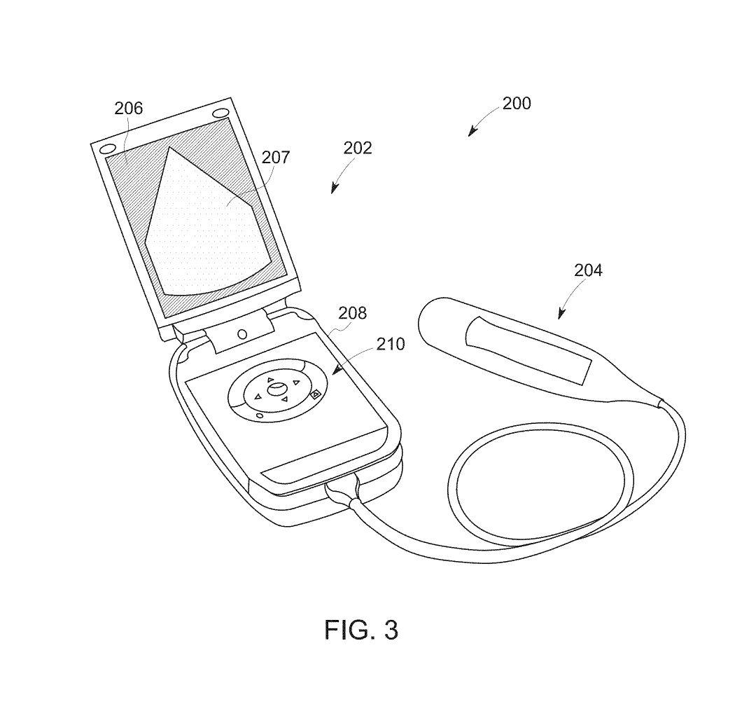

FIG. 3 illustrates a hand carried or pocket-sized ultrasound imaging system 200. The imaging system 200 includes a user interface 202 and an ultrasound probe 204. In some cases, the system 200 may be referred to as a diagnostic system, such as when the probe 204 is not part of the system 200. The imaging system 200 may also include one or more (or all) of the elements (not shown) that are described above with respect to the imaging system 100. For example, the imaging system 200 may include the SAP electronics 110, the beamformer 121, the transmitter 122, the receiver 128, the beamformer 130, the RF processor 132, the memory 134, and the controller circuit 136, which are all shown above in FIG. 1. The controller circuit may be similar to the controller circuit 136 as shown and described with respect to FIG. 2.

The user interface 202 includes a user screen 206 and an input device 208. The user screen 206 is configured to display a medical image 207. The user screen 206 may also display graphical representations, such as user-selectable elements (e.g., markers, icons, etc.) or textual information about the medical image 207. In the illustrated embodiment, the input device 208 includes a click wheel 210 that is configured to receive touches from the operator. However, in other embodiments, the input device 208 may include other elements that are touched or otherwise activated by the operator. In some embodiments, the user screen 206 is touch-sensitive.

The input device 208 and/or the touch-sensitive user screen 206 enable the operator to select various controls and functions for obtaining a medical image and/or analyzing a medical image. For example, the input device 208 and/or the touch-sensitive user screen 206 may enable an operator to select a marker on the user screen 206, move the marker to a corresponding location on the medical image 207, and command the user interface 202 to show a zoom frame. The input device 208 and/or the touch-sensitive user screen 206 may also enable the operator to move the marker within the zoom frame as described below.

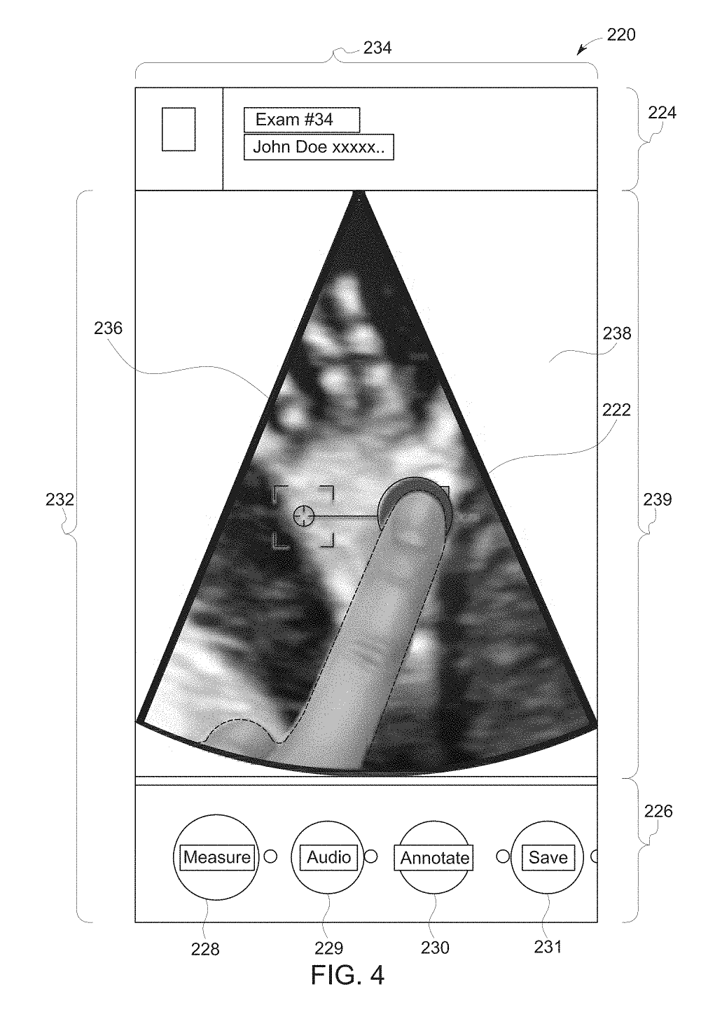

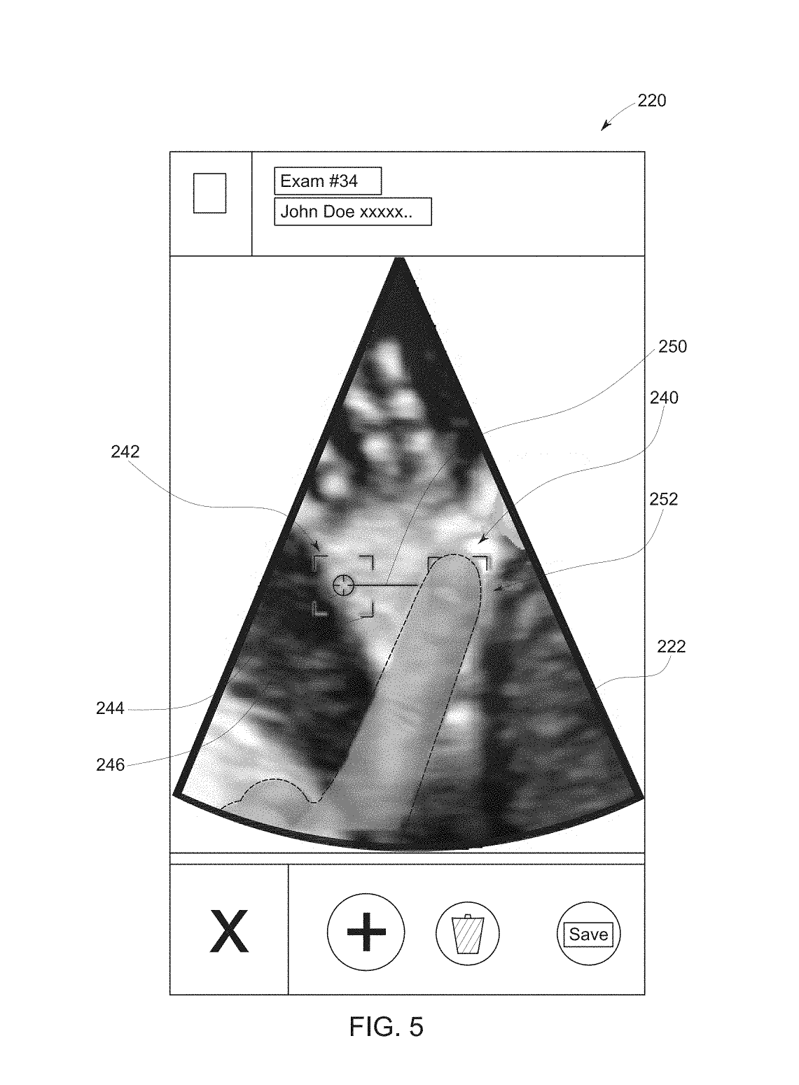

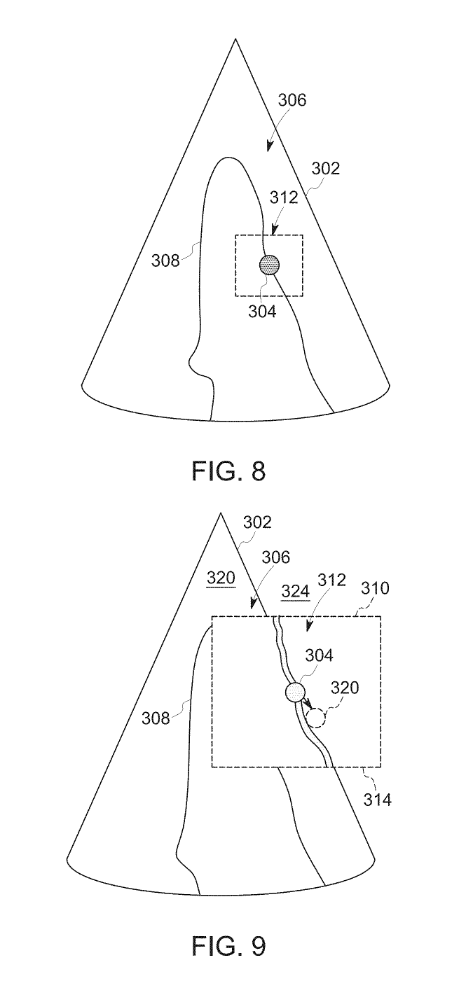

FIG. 4 illustrates a working window 220 that is configured to show a medical image 222 to an operator of a diagnostic system (not shown), such as the medical imaging system 100 (FIG. 1) or the medical imaging system 200 (FIG. 3). In FIG. 4 and also FIGS. 5-7, an individual's hand is shown with the pointer finger pressed against a designated spot. It should be understood that other embodiments may not show an individual's hand. For example, FIGS. 8-11 illustrate an embodiment in which the hand is not shown. The working window 220 may be generated by a controller circuit, such as the controller circuit 136. The working window 220 may be defined by an array of pixels of the user screen (not shown) that are illuminated in a designated manner by the controller circuit to present information to the operator. In some embodiments, the working window 220 covers an entirety of the user screen (not show), such as the user screen 206 (FIG. 3). In other embodiments, however, the working window 220 only covers a portion of the user screen. As shown, the working window 220 includes an information section 224, a control section 226, and a display area 239 therebetween. The control section 226 may include one or more user-selectable elements 228-231. The user-selectable elements 228-231 may be activated by touch or, in some embodiments, by an input device (not shown), such as a mouse or click wheel. The control section 226 is configured to display different user-selectable elements based on a stage of the workflow being carried out by the operator.

The working window 220 has a height (or first dimension) 232 and a width (or second dimension) 234. The working window 220 includes an image area 236 where the medical image 222 is displayed at a predetermined magnification and a non-image area 238 that does not include the medical image 222 (e.g., the area is devoid of the medical image 222). Collectively, the image area 236 and the non-image area 238 may form a display area 239 of the working window 220. The display area 239 may represent the area of the working window 220 that may include the medical image 222 or enlarged portions of the medical image 222 (e.g., zoom frames) as described below. In the illustrated embodiment, the non-image area 238 surrounds nearly the entire image area 236 except for one sharp point. In other embodiments, the non-image area 238 may surround the image area 236 entirely or extend alongside one or more portions thereof.

When the medical image 222 is displayed in the working window 220, the medical image 222 may be shown in accordance with predetermined image settings, such as a brightness, contrast, gain setting, power, time gain compensation (TGC), resolution, color, magnification, or frequency selection. As described herein, the magnification may be changed and, optionally, other settings of the medical image 222 may be changed within a zoom frame shown in the working window. The zoom window may be at least partially within the image area 236 or at least partially within the non-image area 238.

FIG. 5 illustrates the working window 220 at a measurement stage of the workflow. For the working window 220 to be displayed as shown in FIG. 5, the operator may activate the user-selectable element 228 (FIG. 4) (“Measure”). As shown, the working window 220 includes first and second markers 240, 242 that are displayed over the medical image 222. The first and second markers 240, 242 represent endpoints of a distance to be measured. The first and second markers 240, 242 are configured to be moved along the medical image 222 to corresponding locations in the ROI in order to obtain a measurement of designated anatomical matter. More specifically, the user interface (not shown) may receive user inputs for moving the first marker 240 and/or the second marker 242. The user inputs may be, for example, received through an input device or received through a touch-sensitive screen of the user interface.

In the illustrated embodiment, each of the first and second markers 240, 242 includes a marker point 244 and a marker boundary 246. The marker point 244 includes a circle having linear projections that point to a center of the circle. The marker boundary 246 includes lines that are positioned at corners of a square having the marker point 244 at a center of the square. It should be understood that the first and second markers 240, 242 may be other indicia. For example, the marker point 244 may be crosshairs. The marker boundaries 246 may not be shown. In other embodiments, the marker point 244 may be an enlarged dot.

In the illustrated embodiment, the first and second markers 240, 242 may have a measurement line 250 extending therebetween. More specifically, the measurement line 250 may extend between and couple to each of the marker points 244. In other embodiments, the measurement line 250 may only couple to the squares that are partially outlined by the marker boundaries 246. However, in other embodiments, the measurement line 250 is not shown. In some embodiments, the working window 220 may include textual data 255 adjacent to the first marker 240 and/or the second marker 242. For example, the textual data 255 includes a value of the measurement (“3.00 cm”) between the first and second markers 240, 242.

In FIG. 5, the control section 226 includes different user-selectable elements 251-254, including a CANCEL button 251, an ADD button 252, a DISCARD button 253, and a SAVE button 254. If the CANCEL button 251 is activated, the working window 220 may return to the working window 220 shown in FIG. 4. If the ADD button 252 is activated, an additional pair of first and second markers may appear over the medical image 222. If the SAVE button 254 is activated, the medical image 222 along with any measurement data may be stored. If the DISCARD button 253 is activated, the stored data may be deleted.

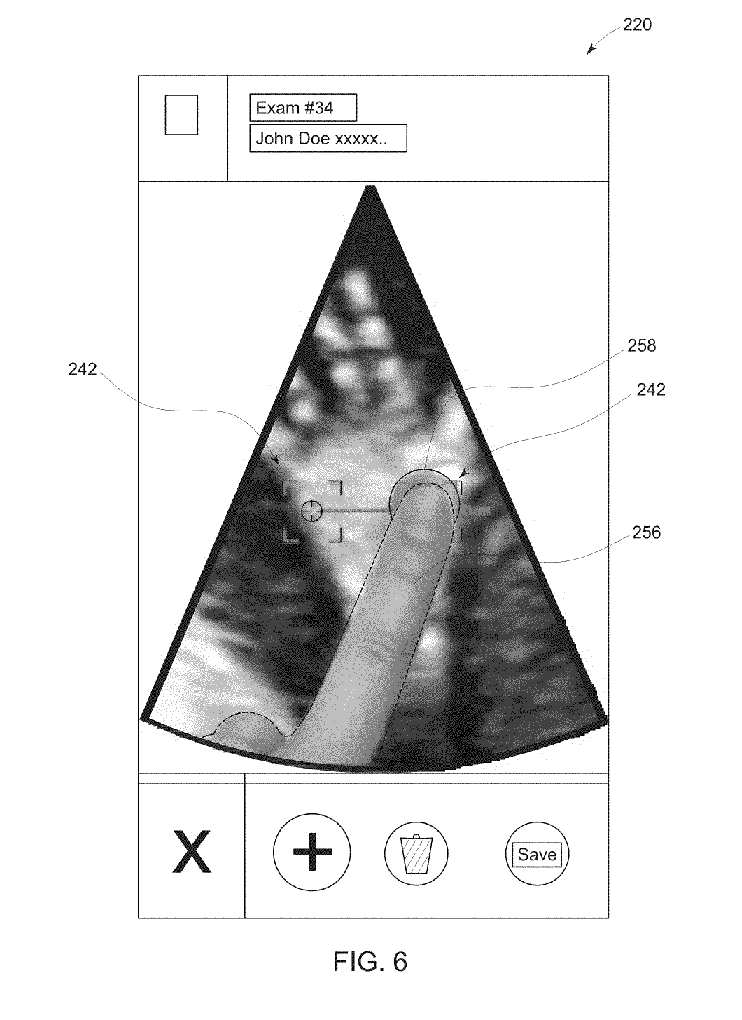

FIG. 6 illustrates the working window 220 when the first marker 240 is activated by a digit 256 (e.g., finger, thumb, or stylus) of the operator. In some embodiments, a graphical representation may appear over the first marker 240. For example, a partially transparent enlarged dot 258 is shown in FIG. 6. In other embodiments, however, the enlarged dot 258 is not shown. In some embodiments, the first marker 240 may change an appearance of the first marker 240 to indicate to the operator that the first marker 240 has been activated. For example, the first marker 240 may at least one of increase in size, change in shape, change in brightness, or change in color when activated. Yet in other embodiments, the first marker 240 does not have any change in appearance when activated. In the illustrated embodiment, the operator slides his or her digit 256 along the surface of the user screen to a corresponding location to move the first marker 240. The second marker 242 may be moved in a similar manner.

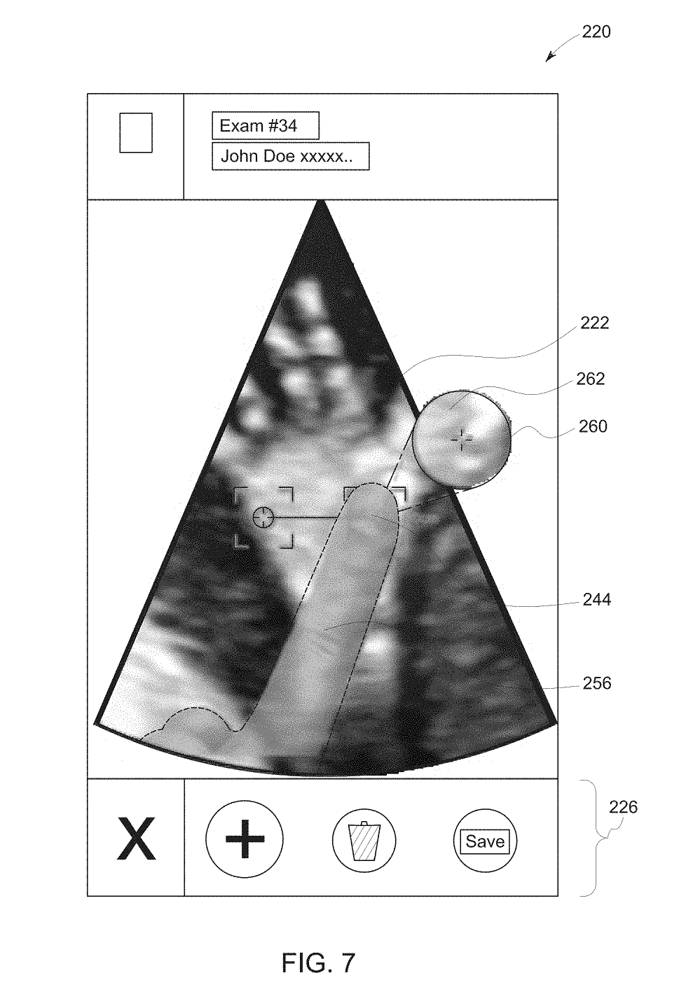

FIG. 7 illustrates the working window 220 having a zoom frame 260 displayed therein. The zoom frame 260 includes a magnified portion of the medical image 222. For example, the marker point 244 surrounds a localized section 262 of the medical image 222. Within the marker point 244, the localized section 262 includes a small portion (e.g., less than 5%) of the medical image 222. The localized section 262, however, is magnified within the zoom frame 260. More specifically, the localized section 262 may have a section area prior to being displayed in the zoom frame 260 and a magnified frame area after the zoom frame 260 is shown. The section area of the localized section 262 may be defined by the marker point 244. The zoom frame 260 may have a shape that is similar to the marker point 244, but larger than the marker point 244. The magnified frame area of the zoom frame 260 is larger than the section area of the localized section prior to magnification. For example, in FIG. 7, the magnified area is at least twenty times (20X) the area defined by the marker point 244.

The zoom frame 260 appears to be positioned over the image area 236 and/or the non-image area 238. In the illustrated embodiment, the user screen is a two-dimensional surface such that the zoom frame 260 and the image area or the non-image area cannot be positioned spatially relative to each other such that one or the other is closer to the operator. It is understood that when a graphical element or representation is described as being positioned over, on top of, behind, below, and like terms, the graphical elements only appear to be positioned as such. Instead, the pixels of the user screen are configured to provide this appearance. For example, when the image area appears to be located under the zoom frame 260, the pixels in the user screen are instructed to modify the corresponding light intensities to provide the appearance that the zoom frame 260 is located over the elements of the image area.

The zoom frame 260 may be activated by one or more user commands. In an exemplary embodiment, the digit 256 of the operator may press the surface of the user screen at the first marker 240 for a designated amount of time (e.g., one or two seconds). In other embodiments, the digit 256 of the operator may repeatedly press the first marker 240. For example, the digit 256 may rapidly press the first marker 240 (e.g., like double clicking with a mouse cursor). In other embodiments, a separate user-selectable element may be provided in the working window 220 that, when activated, may cause the zoom frame 260 to appear. The user-selectable element may be positioned, for example, within the control section 226 or adjacent to the first marker 240.

After the zoom frame 260 is activated, the zoom frame 260 may remain in the working window 220 until the zoom frame 260 is deactivated. Deactivation may occur in a similar manner as described above with respect to activation. For example, the digit 256 may press and hold the zoom frame 260 for a designated period of time or the digit 256 may repeatedly press the zoom frame 260. In other embodiments, the digit 256 may press an area of the working window 220 that is outside of the zoom frame 260 to remove the zoom frame 260. As yet another example, a user-selectable element (not shown) may be presented that, once pressed, may deactivate the zoom frame 260.

While the zoom frame 260 is displayed in the working window 220, the operator may be permitted to move the first marker 240 within the zoom frame 260 to a new location with respect to the ROI. In FIG. 7, the first marker 240 has changed in appearance and is shown as crosshairs. After the first marker 240 is moved to the new location, the zoom frame 260 may be deactivated by the operator.

FIG. 8 illustrates the process of selecting a corresponding location for measuring anatomical matter in greater detail. FIG. 8 illustrates a medical image 302 having a marker 304 positioned thereon. The marker 304 may be similar or identical to the first marker 240 and/or the second marker 242 (FIG. 5). Only a single marker 304 is shown in FIG. 8. In some embodiments, the first marker may be presented individually and positioned by the operator before the second marker is shown. In other embodiments, the first and second markers may be presented simultaneously, such as in FIG. 5. The medical image 302 includes an ROI 306 of the patient that includes anatomical matter 308. The marker 304 has a corresponding location in the medical image 302. The corresponding location may be identified by, for example, Cartesian coordinates or pixel addresses in the image data. The corresponding location may be stored within memory of the diagnostic system.

In order to present a zoom frame 310 (shown in FIG. 9), embodiments may identify a localized section 312 to magnify. For example, when the processor receives instructions to present the zoom frame 310, the processor may designate the localized section 312 of the medical image 302. More specifically, the processor may determine an area of the medical image 302 that is to be magnified within the zoom frame 310. The localized section 312 is represented by dashed lines in FIG. 8. It should be understood that the dashed lines are not required to be shown to the operator. For example, after the zoom frame 310 is activated, the working window may immediately show the zoom frame 310 in FIG. 9 without showing the boundaries of the localized section.

The localized section 312 includes the corresponding location of the marker 304. More specifically, the position of the localized section 312 of the medical image 302 is based on the corresponding location of the marker 304. A shape or area of the localized section 312 may be configured by the diagnostic system. For example, the diagnostic system may position a designated shape (e.g., square or rectangle) that has the corresponding location of the marker 304 at a center of the designated shape. The designated shape may be defined by Cartesian coordinates or pixel addresses. The shape may have predetermined size. For example, the predetermined size may be relative to the size of the medical image 302 on the user screen or may be relative to the size of the medical image. In some embodiments, the size of the localized section 312 is selectable and/or reconfigurable by the operator.

FIG. 9 illustrates the zoom frame 310 over the medical image 306. The localized section 312 is magnified within the zoom frame 310. For example, in some embodiments, the magnification of the localized section 312 in the zoom frame 310 may be at least two times (2X) the magnification of the same area in the medical image 302. In certain embodiments, the magnification of the localized section 312 in the zoom frame 310 may be at least five times (5X) or at least ten times (10X) the magnification of the same area in the medical image 302. In more particular embodiments, the magnification of the localized section 312 in the zoom frame 310 may be at least fifteen times (15X) or at least twenty times (20X) the magnification of the same area in the medical image 302.

As shown in FIG. 9, the zoom frame 310 appears over the medical image 306 and the medical image 302. The zoom frame 310 may appear at least partially in an image area 322 of the working window or at least partially in a non-image area 324 of the working window. In FIG. 9, the zoom frame 310 appears in the image area 322 and in the non-image area 324. In some embodiments, the zoom frame 310 may be indicated by a boundary line 314. The boundary line 314 readily distinguishes or separates the zoom frame 310 and the medical image 302.

Optionally, the shape of the boundary line 314 may be reconfigurable as the zoom frame 310 appears in the working window. For example, the operator may increase the aspect ratio, height, or width of the zoom frame 310 to change the amount of area covered by the localized section 312. In some embodiments, the zoom frame 310 may have different image characteristics than the medical image 302 or the user may be enabled to change the image characteristics of the localized section 312 within the zoom frame 310. For example, the localized section 312 of the medical image 302 in the zoom frame 310 may have a different brightness or contrast.

As shown in FIG. 9, the marker 304 may be moved within the zoom frame 310 to a new location (indicated by dashed circle 320). The imaging system may store (e.g., in memory) the new location of the marker 304 within the medical image 302. After locating the marker 304 at the new location, the operator may remove the zoom frame 310. When the zoom frame 310 is removed, the marker 304 may be positioned at the new location on the medical image 302. Due to the decrease in magnification, the new location may appear only a small distance away from the initial location. In some embodiments, the changed location of the marker 304 in the medical image 302 at the standard magnification may not be noticeable. After both first and second markers are identified, the processor is configured to measure a distance between the locations of the first and second markers.

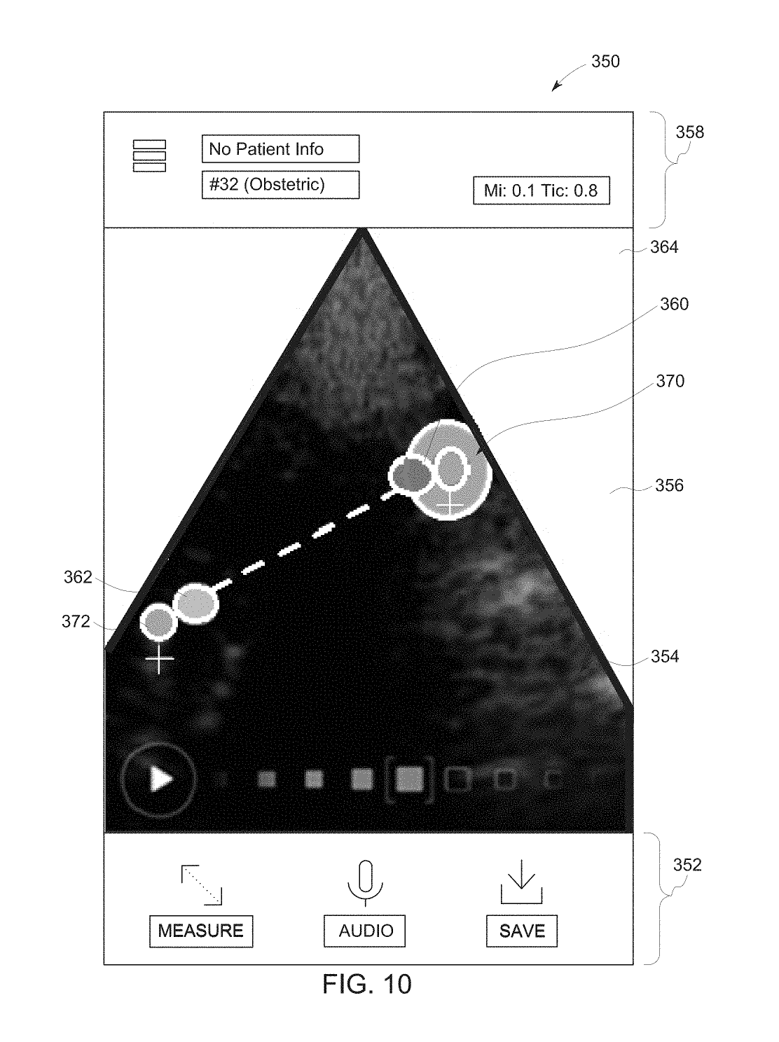

FIG. 10 shows a working window 350 in accordance with an embodiment. The working window 350 may be similar to the working window 220 (FIG. 4). For example, the working window 350 includes a control section 352, an image area 354, a non-image area 356, and an information section 358. The working window 350 also includes first and second markers 360, 362 that are positioned over a medical image 364.

In the illustrated embodiment, the working window 350 is configured to display first and second zoom icons 370, 372 that are positioned adjacent to the first and second markers 360, 362. The corresponding marker of the first zoom icon 370 is the first marker 360, and the corresponding marker of the second zoom icon 372 is the second marker 362. Each zoom icon may be touching or overlapping the corresponding marker or may be positioned near the corresponding marker such that the zoom icon is associated with the corresponding marker. For example, the zoom icon may be closer to the corresponding marker than the other marker. The zoom icon may be within a designated distance of the corresponding marker, such as within two centimeters, one centimeter, or less than half a centimeter.

In some embodiments, the first and second zoom icons 370, 372 are tethered to the first and second markers 360, 362, respectively. More specifically, the first and second zoom icons 370, 372 may move with the first and second markers 360, 362, respectively, when the first and second markers 360, 362 are moved relative to the medical image 364. For example, the first and second zoom icons 370, 372 may move with the first and second markers 360, 362, respectively, as the first and second zoom icons 370, 372, respectively, are moved along the medical image 364. In other embodiments, however, the first and second zoom icons 370, 372 disappear as the first and second markers 360, 362, respectively, are moved and then re-appear when the first and second markers 360, 362, respectively, are positioned at the corresponding locations. In each case, the first and second zoom icons 370, 372 are characterized as moving with or being tethered to the first and second markers 360, 362, respectively.

In particular embodiments, the first and second zoom icons 370, 372 are located at a designated direction relative to the first and second markers 360, 362, respectively. For example, the first zoom icon 370 may be located on the side of the first marker 360 that is the same direction in which the first marker 370 was most recently moved. More specifically, as shown in FIG. 10, the first zoom icon 370 is located on the right-side of the first marker 360. The first zoom icon 370 may be positioned on the right-side of the first marker 360 as the first marker 360 is moved in the rightward direction or after the first marker 360 has been positioned at the corresponding location after moving in the rightward direction. In such embodiments, the operator of the diagnostic system may know that the zoom frame may be activated by quickly lifting his or her digit (not shown) and moving the digit only a short distance in the same direction that the digit was just moved.

In other embodiments, the zoom icon may always appear at a designated position relative to the corresponding marker. For example, the zoom icon may always appear at a 3 o'clock position (or 90°) with respect to the corresponding marker regardless of the location of the corresponding marker. The other zoom icon may always appear at a 9 o'clock position (or 270°) with respect to the corresponding marker regardless of the location of the corresponding marker.

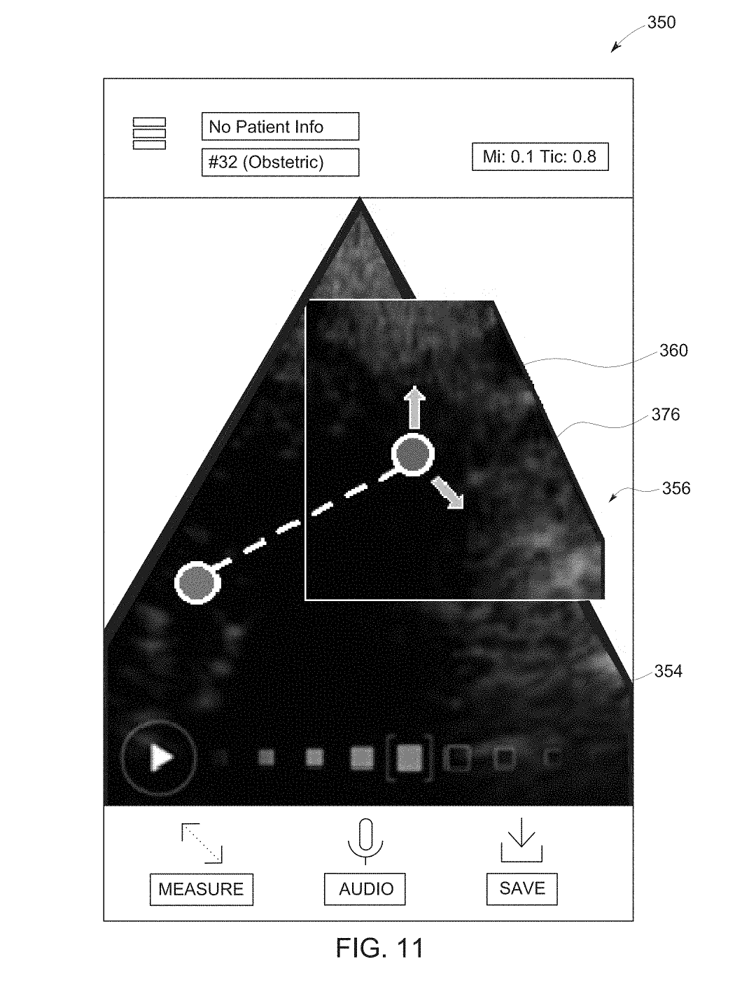

FIG. 11 illustrates a zoom frame 374 after the first zoom icon 370 (FIG. 10) was pressed by the digit (not shown) of the operator. The zoom frame 374 is delineated by a boundary line 376, which has a polygonal shape in FIG. 11. The zoom frame 374 is located partially over the medical image 364 (or image area 354) and partially over the non-image area 356. As described above, the operator is permitted to move the first marker 360 within the zoom frame 374 to a new location relative to the ROI or the medical image 364. In FIG. 11, the first marker 360 has the same appearance in the zoom frame 374 as the appearance of the first marker 360 prior to the zoom frame 374 being activated. In other embodiments, however, the first marker 360 may have a different appearance within the zoom frame 374. After the first marker 360 has been moved to the new location, the zoom frame 374 may be deactivated as described above.

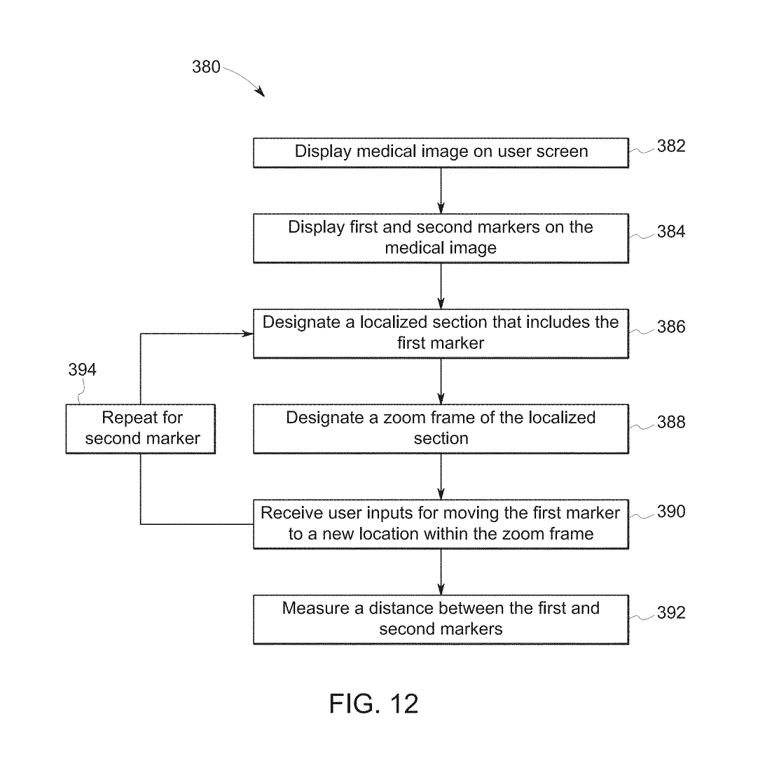

FIG. 12 is a flowchart that illustrates a method of obtaining a measurement of anatomical matter from a medical image. The method 380 includes displaying, at 382, a medical image of an ROI of a patient on a user screen. The medical image may be an ultrasound image, CT image, PET image, MRI image, x-ray image, or an image acquired through another imaging modality. In some embodiments, the medical image is a composite image that was constructed from image data from multiple imaging modalities. In particular embodiments, the user screen is relatively small. For example, the user screen may be the display area of a table computer or a smartphone-like device.

The method 380 also includes displaying, at 384, first and second markers on the medical image. The first and second markers represent endpoints of a distance to be measured in the ROI. The first and second markers have corresponding locations in the ROI or with respect to the medical image. For example, the corresponding location of the corresponding marker may be based on an array of pixels that provide the appearance of the corresponding marker. More specifically, each pixel may have an address within the medical image. The corresponding location may be based on the addresses of the pixels that indicate the marker. In other embodiments, the corresponding location of a corresponding marker may be represented by the address of the pixel located at the center of the marker.

At 386, a localized section of the medical image may be designated. The localized section includes the corresponding location of the first marker. The localized section includes a predetermined amount of the area that surrounds the corresponding location. The predetermined amount of area may be based on the imaging protocol being used. For example, if the protocol requires positioning the marker at a location along an anatomical wall, the localized section may be elongated in a direction that extends parallel to the wall. In other embodiments, the localized section has a predetermined shape, such as circular, rectangular, or other polygonal shape. The operation of designating the localized section, at 386, may be initiated by a command from the operator. For example, the operator may press on the first marker in a designated manner or may press a zoom icon.

The method 380 also includes displaying, at 388, a zoom frame of the localized section over the medical image. The localized section may be magnified within the zoom frame. At 390, the method includes receiving user inputs to move the first marker within the zoom frame to a new location in the ROI. For example, the first marker may appear at approximately a central location of the zoom frame. The operator may press the first marker and move the first marker to the new location. An address of the new location may be stored. Operations 386, 388, 390 may be repeated for the second marker and/or the first marker again. At 392, a distance between the corresponding location of the second marker and the new location of the first marker may be measured. The corresponding location of the second marker may be a new location that was identified through a zoom frame.

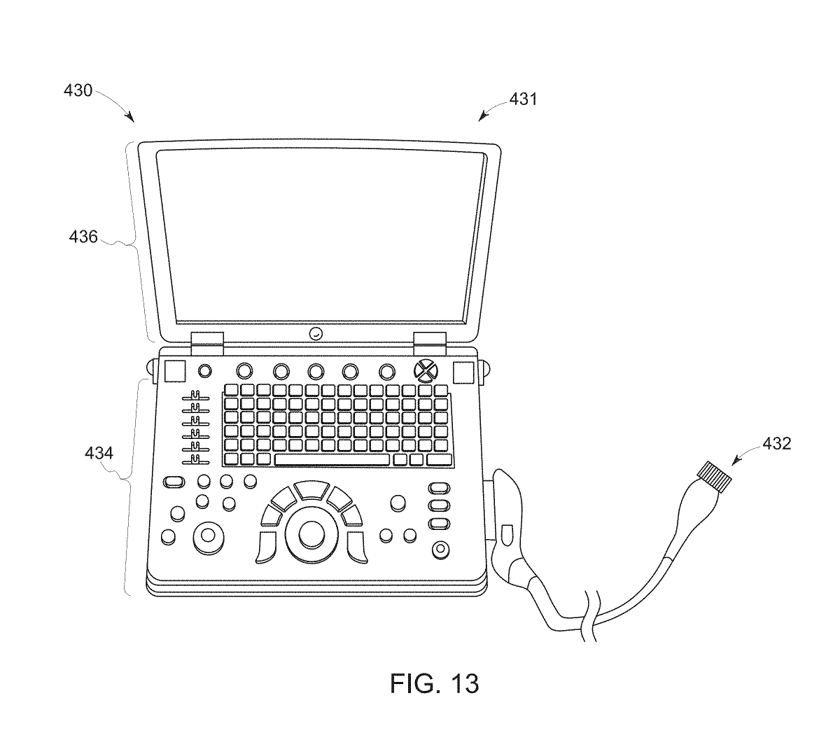

FIG. 13 illustrates an ultrasound imaging system 430 having a probe 432 that may be configured to acquire 3D ultrasonic data or multi-plane ultrasonic data. The imaging system 430 also includes a computer 431, which may constitute or include the user interface of the imaging system 430. For embodiments that do not include the probe 432, the imaging system 430 may be referred to as a diagnostic system. The probe 432 may have a 2D array of elements as discussed previously with respect to the probe. The computer 431 is a laptop or notebook computer that includes an input device 434 and a user screen 436. The input device 434 and, optionally, the user screen 436 are configured to receive user inputs. The user screen 436 may display one or more working windows having a medical image therein. The computer 431 may enable the operator to move markers along the medical image and activate zoom frames as described above.

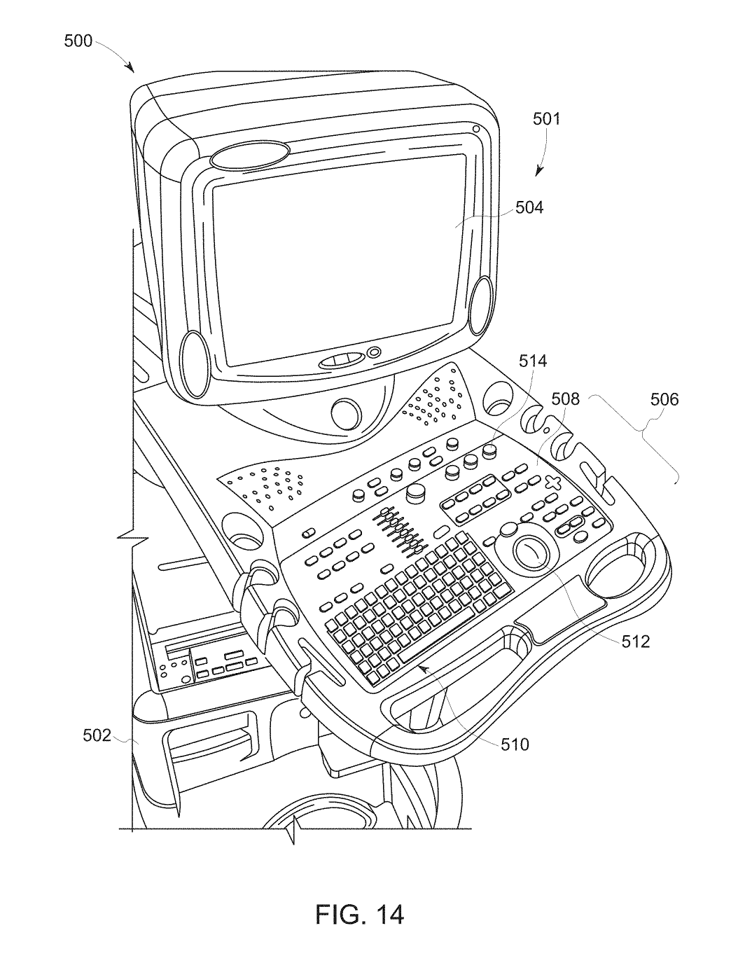

FIG. 14 illustrates an ultrasound imaging system 500 provided on a movable base 502. The portable ultrasound imaging system 500 may also be referred to as a cart-based system. The imaging system 500 includes a user interface 501 having a display 504 and an input device 506. The display 504 may be separate or separable from the input device 506. The input device 506 may optionally be a touchscreen, allowing the operator to select options by touching displayed graphics, icons, and the like.

The input device 506 also includes control buttons 508 that may be used to control the portable ultrasound imaging system 500 as desired or needed, and/or as typically provided. The input device 506 provides multiple interface options that the user may physically manipulate to interact with ultrasound data and other data that may be displayed, as well as to input information and set and change scanning parameters and viewing angles, etc. For example, a keyboard 510, trackball 512 and/or multi-function controls 514 may be provided.

As described above, the various components and modules described herein may be implemented as part of one or more computers or processors. The computer or processor may include a microprocessor. The microprocessor may be connected to a communication bus. The computer or processor may also include a memory. The memory may include Random Access Memory (RAM) and Read Only Memory (ROM). The computer or processor further may include a storage system or device, which may be a hard disk drive or a removable storage drive such as a floppy disk drive, optical disk drive, and the like. The storage system may also be other similar means for loading computer programs or other instructions into the computer or processor. The instructions may be stored on a tangible and/or non-transitory computer readable storage medium coupled to one or more servers.

As used herein, the term “computer” or “computing system” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer” or “computing system.”

The set of instructions may include various commands that instruct the computer or processor as a processing machine to perform specific operations such as the methods and processes described herein. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine. The program is compiled to run on both 32-bit and 64-bit operating systems. A 32-bit operating system like Windows XP™ can only use up to 3 GB bytes of memory, while a 64-bit operating system like Window's Vista™ can use as many as 16 exabytes (16 billion GB).

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A medical diagnostic system comprising:
a user interface having a user screen that is configured to display a medical image to an operator of the diagnostic system, the medical image including a region-of-interest (ROI) of a patient, wherein the user interface is configured to show first and second markers on the medical image, the first and second markers representing endpoints of a distance to be measured, the user interface being configured to receive user inputs to move the first marker or the second marker to corresponding locations in the ROI; and
a controller configured to designate a localized section of the medical image that includes the corresponding location of the first marker, the controller configured to display a zoom frame of the localized section over the medical image, the localized section being magnified within the zoom frame;
wherein the user interface is configured to receive user inputs to move the first marker within the zoom frame to a new location with respect to the ROI within the zoom frame, the controller configured to measure the distance between the corresponding location of the second marker and the new location of the first marker.

2. The diagnostic system of claim 1, wherein the localized section has a section area prior to being displayed in the zoom frame and has a frame area when displayed in the zoom frame, the frame area being at least two times the section area.

3. The diagnostic system of claim 1, wherein the controller displays the zoom frame in response to a user command to display the zoom frame.

4. The diagnostic system of claim 1, wherein the user interface is configured to display a zoom icon on the user screen that is associated with the first marker, the controller displaying the zoom frame in response to the zoom icon being activated.

5. The diagnostic system of claim 4, wherein the zoom icon is tethered to the first marker such that the zoom icon moves with the first marker.

6. The diagnostic system of claim 1, wherein the user interface is configured to remove the zoom frame, the first marker having the new location with respect to the ROI after the zoom frame is removed.

7. The diagnostic system of claim 1, wherein the localized section is a first localized section, the controller being configured to designate a second localized section of the medical image that includes the corresponding location of the second marker and display a zoom frame of the second localized section over the medical image, wherein the user interface is configured to receive user inputs to move the second marker within the zoom frame of the second localized section to a new location in the ROI within the zoom frame of the second localized section, the controller configured to measure the distance between the new location of the second marker and the new location of the first marker.

8. The diagnostic system of claim 1, wherein the diagnostic system includes a handheld device or portable computer that has the user screen.

9. A method comprising:
displaying first and second markers on a medical image that includes a region-of-interest (ROI) of a patient, the first and second markers representing endpoints of a distance to be measured in the ROI, the first and second markers having corresponding locations in the ROI;
designating a localized section of the medical image that includes the corresponding location of the first marker;
displaying a zoom frame of the localized section over the medical image, the localized section being magnified within the zoom frame;
receiving user inputs to move the first marker within the zoom frame to a new location in the ROI; and
measuring a distance between the corresponding location of the second marker and the new location of the first marker.

10. The method of claim 9, wherein the localized section has a section area prior to being displayed in the zoom frame and has a frame area when displayed in the zoom frame, the frame area being at least two times the section area.

11. The method of claim 9, wherein displaying the zoom frame occurs in response to a user command to display the zoom frame.

12. The method of claim 9, further comprising displaying a zoom icon on the user screen that is associated with the first marker, wherein displaying the zoom frame occurs in response to the zoom icon being activated.

13. The method of claim 9, wherein the zoom icon is tethered to the first marker such that the zoom icon moves with the first marker.

14. The method of claim 9, wherein the user interface is configured to remove the zoom frame, the first marker having the new location in the ROI after the zoom frame is removed.

15. The medical imaging system comprising:
a medical imager configured to acquire a medical image of a region-of-interest (ROI) of a patient;
a user interface having a user screen that is configured to display the medical image to an operator of the medical imaging system, wherein the user interface is configured to display first and second markers on the medical image, the first and second markers representing endpoints of a distance to be measured, the user interface being configured to receive user inputs to move the first marker or the second marker to corresponding locations in the ROI; and
a controller configured to designate a localized section of the medical image that includes the corresponding location of the first marker, the controller configured to display a zoom frame of the localized section over the medical image, the localized section being magnified within the zoom frame;
wherein the user interface is configured to receive user inputs to move the first marker within the zoom frame to a new location in the ROI within the zoom frame, the controller configured to measure the distance between the corresponding location of the second marker and the new location of the first marker.

16. The medical imaging system of claim 15, wherein the medical imager includes an ultrasound probe, the medical image being an ultrasound image.

17. The medical imaging system of claim 16, wherein the medical imaging system includes a handheld device or portable computer that has the user screen, the ultrasound probe being communicatively coupled to the handheld device or the portable computer.

18. The medical imaging system of claim 15, wherein the localized section has a section area prior to being displayed in the zoom frame and has a frame area when displayed in the zoom frame, the frame area being at least two times the section area.

19. The medical imaging system of claim 15, wherein the user interface is configured to display a zoom icon on the user screen that is associated with the first marker, the controller displaying the zoom frame in response to the zoom icon being activated.

20. The diagnostic system of claim 19, wherein the zoom icon is tethered to the first marker such that the zoom icon moves with the first marker.
